(19) United States
(12) Patent Application Publication
(10) Pub. No.: US 2022/0142594 A1
(43) Pub. Date: May 12, 2022

(54) POSITRON EMISSION TOMOGRAPHY APPARATUS

(71) Applicant: CANON MEDICAL SYSTEMS CORPORATION, Tochigi (JP)

(72) Inventor: Takayuki YAMAZAKI, Nasushiobara (JP)

(73) Assignee: CANON MEDICAL SYSTEMS CORPORATION, Tochigi (JP)

(21) Appl. No.: 17/487,504

(22) Filed: Sep. 28, 2021

(30) Foreign Application Priority Data
Nov. 11, 2020 (JP) 2020-187701

(51) Int. Cl. A61B 6/00; A61B 6/03; A61B 6/04; G01T 1/29

(52) U.S. Cl. CPC A61B 6/4275; A61B 6/037; A61B 6/0407; G01T 1/2985; A61B 6/54; A61B 6/5264; A61B 6/4266

(57) ABSTRACT

A positron emission tomography (PET) apparatus according to an embodiment includes a PET detector and processing circuitry. The PET detector includes a detector ring configured with a plurality of detector modules arranged in an annular shape. The processing circuitry is configured to acquire information regarding a scan mode of a PET scan for a subject. The processing circuitry is configured to control a relative position of the detector modules in an axial direction of the detector ring based on the information.

### CROSS-REFERENCE TO RELATED APPLICATIONS

This application is based upon and claims the benefit of priority from Japanese Patent Application No. 2020-187701, filed on Nov. 11, 2020; the entire contents of which are incorporated herein by reference.

### FIELD

Embodiments described herein relate generally to a Positron Emission Tomography (PET) apparatus.

### BACKGROUND

Conventionally, in a PET apparatus in general, the position of a PET detector formed in an annular shape is fixed and, when an area exceeding an Axial Field Of View (AFOV) defined by the length in the axial direction of the PET detector is imaged, imaging is performed for a plurality of times while moving a couchtop on which a subject is placed in the axial direction of the PET detector. Therefore, for example, when a wide area is imaged as in a case of whole-body imaging, it is required to perform imaging for a plurality of times and to have a specific length of imaging time for each imaging. Thus, it is difficult to shorten the entire imaging time and to improve the imaging throughput.

### BRIEF DESCRIPTION OF THE DRAWINGS

FIG. 1 is a diagram illustrating a configuration example of a PET apparatus according to a first embodiment;

FIG. 2 is a diagram illustrating a configuration of a PET detector according to the first embodiment;

FIG. 3 is a diagram illustrating an example of relative position control of detector modules performed by a control function according to the first embodiment;

FIG. 4 is a diagram illustrating an example of relative position control of the detector modules performed by the control function according to the first embodiment;

FIG. 5 is a diagram illustrating an example of relative position control of the detector modules performed by the control function according to the first embodiment;

FIG. 6 is a diagram illustrating an example of relative position control of the detector modules performed by the control function according to the first embodiment;

FIG. 7 is a diagram illustrating an example of imaging of a whole body performed by the control function according to the first embodiment;

FIG. 8 is a flowchart illustrating an order of processing executed by processing circuitry of the PET apparatus according to the first embodiment;

FIG. 9 is a diagram illustrating an example of relative position control of detector modules performed by a control function according to a second embodiment;

FIG. 10 is a diagram illustrating an example of relative position control of the detector modules performed by the control function according to the second embodiment; and

FIG. 11 is a diagram illustrating an example of relative position control of the detector modules performed by the control function according to the second embodiment.

### DETAILED DESCRIPTION

A PET apparatus according to embodiments includes a PET detector, an acquisition unit, and a control unit. The PET detector includes a detector ring configured with a plurality of detector modules arranged in an annular shape. The acquisition unit is configured to acquire information regarding a scan mode of a PET scan performed on a subject. The control unit is configured to control the relative position of the detector modules in the axial direction of the detector ring based on the information.

Hereinafter, the embodiments of the PET apparatus will be described in detail by referring to the accompanying drawings.

### First Embodiment

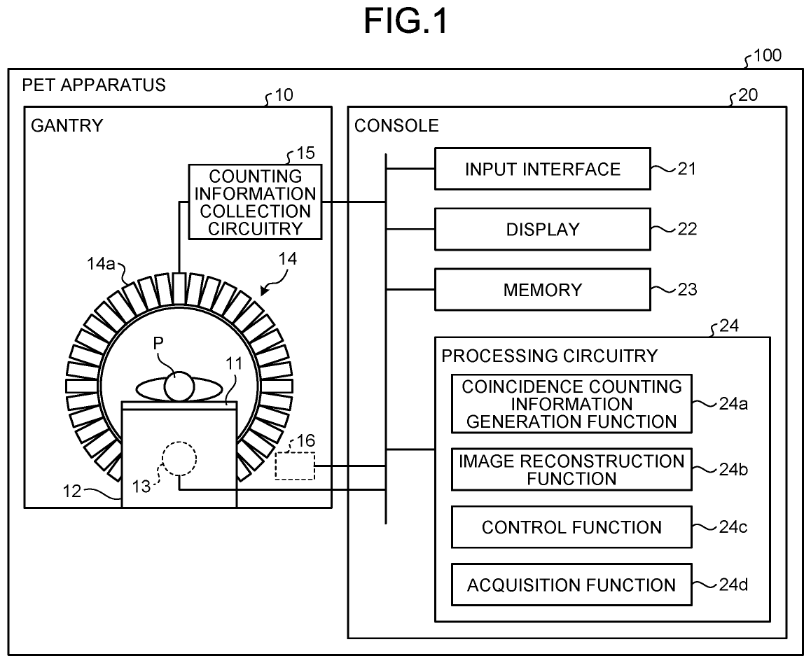

FIG. 1 is a diagram illustrating a configuration example of the PET apparatus according to the first embodiment.

For example, as illustrated in FIG. 1, a PET apparatus 100 according to the embodiment includes a gantry 10 and a console 20.

The gantry 10 detects, when imaging of the subject P is performed, pair annihilation gamma rays that are emitted when positrons emitted from a tracer given to a subject P annihilate with electrons, and counts the detected pair annihilation gamma rays to collect counting information. Note that the gantry 10 includes an opening part formed to go through the gantry 10 in a horizontal direction so as to place the subject P in the opening part at the time of imaging.

Specifically, the gantry 10 includes a couchtop 11, a couch 12, a couch drive mechanism 13, a PET detector 14, counting information collection circuitry 15, and a detector drive mechanism 16.

The couchtop 11 is a bed on which the subject P is placed. The couch 12 supports the couchtop 11 to be movable in the horizontal direction, and moves the couchtop 11 on which the subject P is placed toward the opening part of the gantry 10 at the time of imaging. The couch drive mechanism 13 moves the couchtop 11 that is supported by the couch 12.

The PET detector 14 detects and counts pair annihilation gamma rays emitted from the subject P, converts the detected pair annihilation gamma rays to electric signals, and outputs those. Specifically, the PET detector 14 is formed by arranging a plurality of detector units 14a in an annular shape, and it is disposed to surround the opening part formed in the gantry 10. Each of the detector units 14a included in the PET detector 14 counts and detects the pair annihilation gamma rays emitted from the subject P placed in the opening part of the gantry 10.

The counting information collection circuitry 15 collects counting information on the pair annihilation gamma rays based on the electric signals output from the PET detector 14. Specifically, the counting information collection circuitry 15 converts the electric signals output from the PET detector 14 into digital signals, and generates a list of counting information including detected positions of the pair annihilation gamma rays, energy values, and detected time. Then, the counting information collection circuitry 15 stores the generated list to a memory 23.

Note that the detector drive mechanism 16 will be described in detail later.

The console 20 receives various kinds of operations for the PET apparatus 100 from an operator, and controls actions of the PET apparatus 100 based on the received operations.

Specifically, the console 20 includes an input interface 21, a display 22, processing circuitry 24, the memory 23, a coincidence counting information generation function 24a, an image reconstruction function 24b, a control function 24c, and an acquisition function 24d. Note that each of the units provided to the console 20 is connected via a bus. While a case where the gantry 10 and the console 20 are separate bodies is described as an example, the console 20 or a part of the structural elements of the console 20 may be included in the gantry 10.

The input interface 21 receives various kinds of input operations from the operator, converts the received input operations into electric signals, and outputs those to the processing circuitry 24. For example, the input interface 21 is implemented by a mouse, a keyboard, a trackball, a switch, a button, a joystick, a touchpad with which an input operation is performed by touching an operation screen, a touch screen in which a display screen and a touch pad are integrated, noncontact input circuitry using an optical sensor, voice input circuitry or the like for performing setting and the like of imaging conditions and Region Of Interest (ROI). For example, the input interface 21 may be provided to the gantry 10. For example, the input interface 21 may be configured with a tablet terminal or the like capable of having wireless communication with the main body of the console 20. Furthermore, the input interface 21 is not limited to a type that includes physical operation components such as a mouse, a keyboard, and the like. For example, as the input interface 21, there is also electric signal processing circuitry that receives electric signals corresponding to input operations from an external input device provided separately from the console 20 and outputs the electric signals to the processing circuitry 24.

The display 22 displays various kinds of information. For example, the display 22 outputs PET images generated by the processing circuitry 24 and a Graphical User Interface (GUI) or the like for receiving various kinds of operations from the operator. For example, the display 22 is a liquid crystal display or a Cathode Ray Tube (CRT) display. For example, the display 22 may be provided in the gantry 10. Furthermore, the display 22 may be a desktop type or may be configured with a tablet terminal or the like capable of having wireless communication with the console 20.

The memory 23 stores various kinds of data used in the PET apparatus 100. For example, the memory 23 is implemented by a Random Access memory (RAM), a semiconductor memory element such as a flash memory, a hard disk, an optical disc, or the like.

The processing circuitry 24 controls the actions of the entire PET apparatus 100. Specifically, the processing circuitry 24 includes the coincidence counting information generation function 24a, the image reconstruction function 24b, the control function 24c, and the acquisition function 24d.

For example, the processing circuitry 24 is implemented by a processor. In that case, the processing functions of the processing circuitry 24 are stored in the memory 23 in a form of computer programs that can be executed by a computer. Furthermore, the processing circuitry 24 reads out and executes each of the computer programs from the memory 23 to implement the processing functions corresponding to the respective computer programs. In other words, the processing circuitry 24 comes to have each of the processing functions illustrated in the processing circuitry 24 of FIG. 1 when reading out each of the computer programs.

The coincidence counting information generation function 24a generates a time series list of coincidence counting information by using the counting information collected by the counting information collection circuitry 15. Specifically, the coincidence counting information generation function 24a retrieves sets of counting information regarding the pair annihilation gamma rays counted almost simultaneously from the counting information stored in the memory 23 based on the detected time of the counting information. Then, the coincidence counting information generation function 24a generates coincidence counting information for each retrieved set of the counting information, and stores the generated coincidence counting information in the memory 23 in a time series order.

The image reconstruction function 24b reconstructs a PET image based on the time series list of the coincidence counting information generated by the coincidence counting information generation function 24a. Specifically, the image reconstruction function 24b reads out the time series list of the coincidence counting information stored in the memory 23, and reconstructs the PET image by using the read-out time series list. Furthermore, the image reconstruction function 24b stores the reconstructed PET image to the memory 23. The control function 24c controls each of the units of the gantry 10 and the console 20 to perform the overall control of the PET apparatus 100. For example, the control function 24c controls the couch drive mechanism 13 to move the couchtop 11. Furthermore, the control function 24c controls the counting information collection circuitry 15 to collect the counting information on the pair annihilation gamma rays emitted from the subject P, for example.

The acquisition function 24d will be described in detail later.

The configuration example of the PET apparatus 100 according to the first embodiment has been described heretofore.

Note that in a PET apparatus in general, the position of the PET detector formed in an annular shape is fixed and, when an area exceeding an AFOV defined by the length in the axial direction of the PET detector is imaged, imaging is performed for a plurality of times while moving the couchtop on which the subject is placed in the axial direction of the PET detector. Therefore, for example, when a wide area is imaged as in a case of whole-body imaging, it is required to perform imaging for a plurality of times and to have a specific length of imaging time for each imaging. Thus, it is difficult to shorten the entire imaging time and to improve the imaging throughput.

Based on the above, the PET apparatus 100 according to the embodiment is configured to be able to improve the imaging throughput.

Hereinafter, the configuration of the PET apparatus 100 according to the embodiment will be described in a specific manner.

First, in the embodiment, the PET detector 14 includes a detector ring configured with a plurality of detector modules arranged in an annular shape. Note that the detector ring is disposed such that the center axis thereof coincides with that of the PET detector 14.

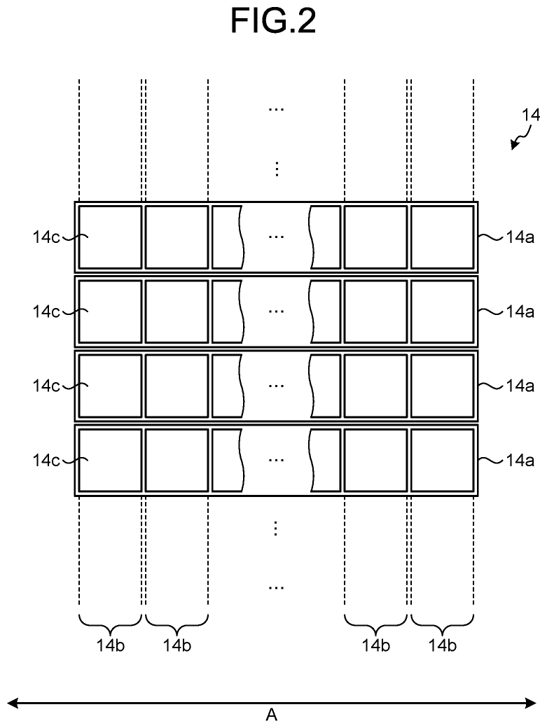

FIG. 2 is a diagram illustrating the configuration of the PET detector 14 according to the first embodiment. Note that FIG. 2 illustrates a state of a part of the PET detector 14 when viewed from a direction orthogonal to the axial direction (direction indicated by arrow A) of the PET detector 14.

For example, as illustrated in FIG. 2, the PET detector 14 includes a plurality of detector rings 14b disposed by being arranged in the axial direction with their respective center axes aligned with each other. Note that the detector rings 14b are configured with the detector units 14a arranged in an annular shape, the detector units 14a each being configured with the detector modules 14c as many as the detector rings 14b and arranged in the axial direction.

Furthermore, each of the detector modules 14c included in the detector unit 14a includes a plurality of detection elements arranged two-dimensionally in the circumferential direction and the axial direction of the PET detector 14, and each of the detection elements counts and detects the pair annihilation gamma rays emitted from the subject P. That is, in the embodiment, the range where the detector modules 14c are disposed in the axial direction of the PET detector 14 is defined as the AFOV.

Furthermore, in the embodiment, the detector drive mechanism 16 individually moves the detector units 14a included in the PET detector 14 in the axial direction of the PET detector 14.

Furthermore, in the embodiment, the acquisition function 24d of the processing circuitry 24 acquires information regarding a scan mode of a PET scan for the subject P. Then, the control function 24c of the processing circuitry 24 controls the relative position of the detector modules 14c in the axial direction of the detector rings based on the information regarding the scan mode acquired by the acquisition function 24d. Note that the acquisition function 24d is an example of the acquisition unit. Furthermore, the control function 24c is an example of the control unit.

Specifically, the control function 24c controls the relative position of the detector units 14a in the axial direction of the detector rings 14b to control the relative position of the detector modules 14c.

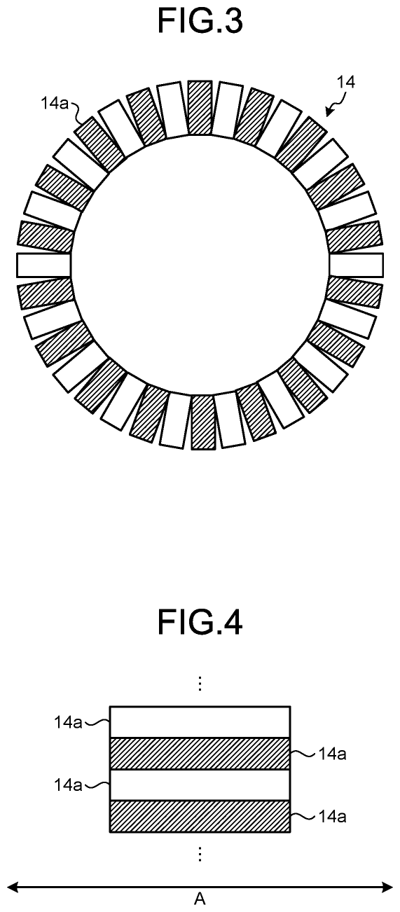

FIG. 3 to FIG. 6 are diagrams illustrating examples of the control of the relative position of the detector modules 14c performed by the control function 24c according to the first embodiment.

For example, as illustrated in FIG. 3, the control function 24c controls the detector drive mechanism 16 to move the detector units 14a included in the PET detector 14 in a unit of two groups in which every other detector unit 14a is assigned in the circumferential direction. In FIG. 3, as for the two groups, the detector units 14a included in the first group are illustrated in white, while the detector units 14a included in the second group are illustrated with hatched pattern.

Specifically, the control function 24c controls the detector drive mechanism 16 to move one of or both of the detector units 14a included in the first group and the detector units 14a included in the second group to the axial direction.

At this time, the control function 24c controls the shift amount of the detector units 14a in the axial direction of the PET detector 14 to change the AFOV or the sensitivity of the PET detector 14 in accordance with the scan mode.

For example, the control function 24c controls the shift amount of the detector units 14a included in the first group and the detector units 14a included in the second group in three modes that are a normal mode, a wide mode, and an intermediate mode.

For example, as illustrated in FIG. 4, the control function 24c in the normal mode controls the detector drive mechanism 16 so that the positions of the detector units 14a included in the first group coincide with the positions of the detector units 14a included in the second group in the axial direction of the PET detector 14. In the normal mode, the AFOV becomes the minimum while the sensitivity per AFOV becomes the maximum.

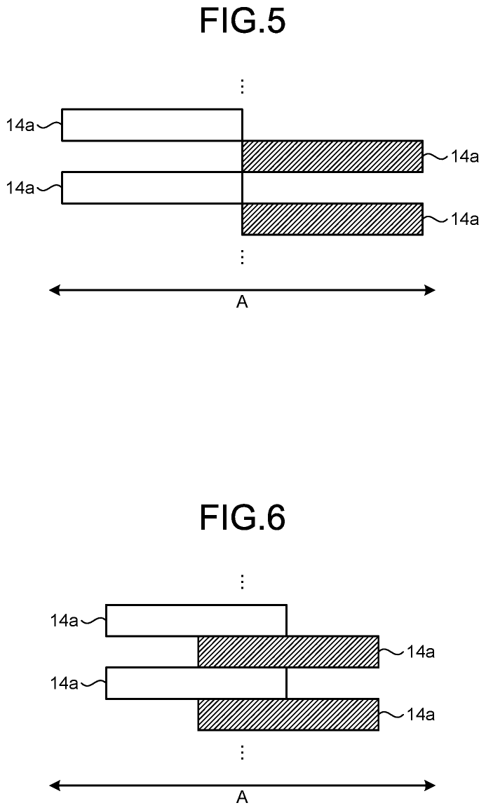

Furthermore, as illustrated in FIG. 5, for example, the control function 24c in the wide mode controls the detector drive mechanism 16 so that the detector units 14a included in the first group and the detector units 14a included in the second group are in a positional relationship not overlapping with each other in the axial direction of the PET detector 14. In the wide mode, the sensitivity per AFOV becomes the minimum while the AFOV becomes the maximum. Furthermore, due to the gap between the detector units, the signal-to-noise ratio may be deteriorated and the incident event that may occur within the gap between the neighboring detector units may be lost.

Furthermore, as illustrated in FIG. 6, for example, the control function 24c in the intermediate mode controls the detector drive mechanism 16 so that the detector units 14a included in the first group and the detector units 14a included in the second group are in a positional relationship partially overlapping with each other in the axial direction of the PET detector 14. In the intermediate mode, the AFOV and the sensitivity per AFOV respectively become about the medium with respect to the normal mode and the wide mode. In the intermediate mode, the shift amount of the detector units 14a may be changed arbitrarily by the operator, for example.

As a way of example, the acquisition function 24d acquires information regarding a body site of the subject P as the information regarding the scan mode, for example. In this case, the control function 24c controls the relative position of the detector modules 14c so that the shift amount of the detector modules 14c in the axial direction of the detector rings 14b changes in accordance with the body site of the subject P.

For example, when imaging of a whole body of the subject P is performed, the acquisition function 24d acquires information indicating a plurality of body sites of the subject P as the information regarding the scan mode. For example, the acquisition function 24d acquires the information regarding the body sites from protocol information set in advance as an imaging condition. Alternatively, for example, the acquisition function 24d may acquire information regarding the body sites input by the operator before imaging is stated.

Then, the control function 24c controls the shift amount of the detector units 14a at respective positions to perform imaging with the AFOV or the sensitivity corresponding to each of the body sites while gradually moving the couchtop 11 on which the subject P is placed in the axial direction of the PET detector 14 for each of the body sites.

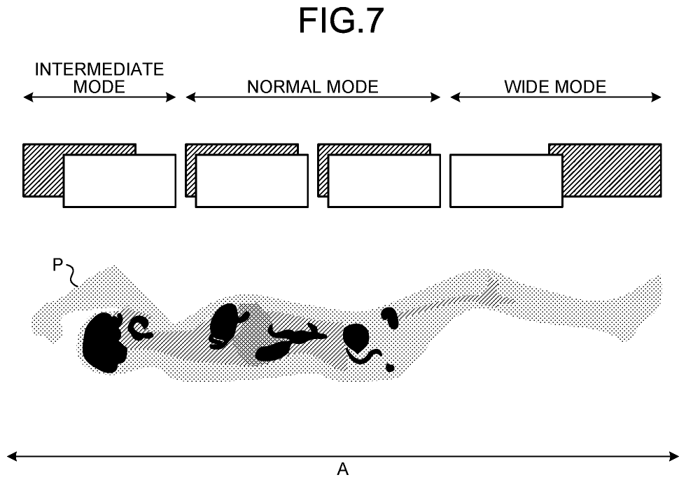

FIG. 7 is a diagram illustrating an example of imaging of a whole body performed by the control function 24c according to the first embodiment.

For example, as illustrated in FIG. 7, when imaging of a whole body of the subject P is performed, the control function 24c controls the shift amount of the detector units 14a so that imaging of the lower limbs is performed with the wide mode, imaging of the body trunk is performed with the normal mode, and imaging of the head is performed with the intermediate mode. Thus, imaging of the lower limbs having a wider area compared to other body sites can be performed with the maximum AFOV, imaging of the body trunk where higher spatial resolution is required compared to other body sites can be performed with the minimum AFOV, and imaging of the head where medium spatial resolution is required can be performed with the middle-size AFOV.

That is, in this example, it is possible to perform imaging with the AFOV adjusted to an appropriate size in accordance with the body site of the subject P.

As another example, the acquisition function 24d may acquire attenuation data indicating a distribution of attenuation coefficients within the body of the subject P as the information regarding the scan mode, for example. In this case, the control function 24c controls the relative position of the detector modules 14c so that the counted number of the pair annihilation gamma rays detected by the PET detector 14 at the respective positions along the axial direction of the detector rings 14b becomes uniform.

For example, when imaging of a whole body of the subject P is performed, the acquisition function 24d acquires the attenuation data indicating the distribution of the attenuation coefficients in the whole body of the subject P as the information regarding the scan mode. For example, the acquisition function 24d acquires, as the attenuation data, an attenuation coefficient map generated from CT images of the same subject P imaged by an X-ray Computed Tomography (CT) apparatus before imaging is performed by the PET apparatus 100. Alternatively, for example, the acquisition function 24d may acquire, as the attenuation data, an attenuation coefficient map generated from PET images of the same subject P imaged by the PET apparatus 100 while moving the couchtop 11 without shifting the detector units 14a before the main imaging is performed.

Then, the control function 24c continuously or gradually moves the couchtop 11 on which the subject P is placed in the axial direction of the PET detector 14 so as to perform control such that the shift amount of the detector units 14a becomes smaller at the position of a larger attenuation coefficient and such that the shift amount of the detector units 14a becomes larger at the position of a smaller attenuation coefficient. Thus, it is possible to adjust the size of the AFOV so that the counted number of the pair annihilation gamma rays detected by the PET detector 14 at the respective positions in the axial direction of the PET detector 14 becomes uniform.

That is, in this example, it is possible to perform imaging with the AFOV adjusted to an appropriate size in accordance with the distribution of the attenuation coefficients in the body of the subject P.

Furthermore, as another example, the acquisition function 24d may receive the shift amount of the detector units 14a from the operator as the information regarding the scan mode. In this case, the control function 24c controls the relative position of the detector modules 14c so as to shift the detector units 14a by the amount received from the operator.

That is, in this example, the operator is enabled to adjust the AFOV to any desired size to perform imaging.

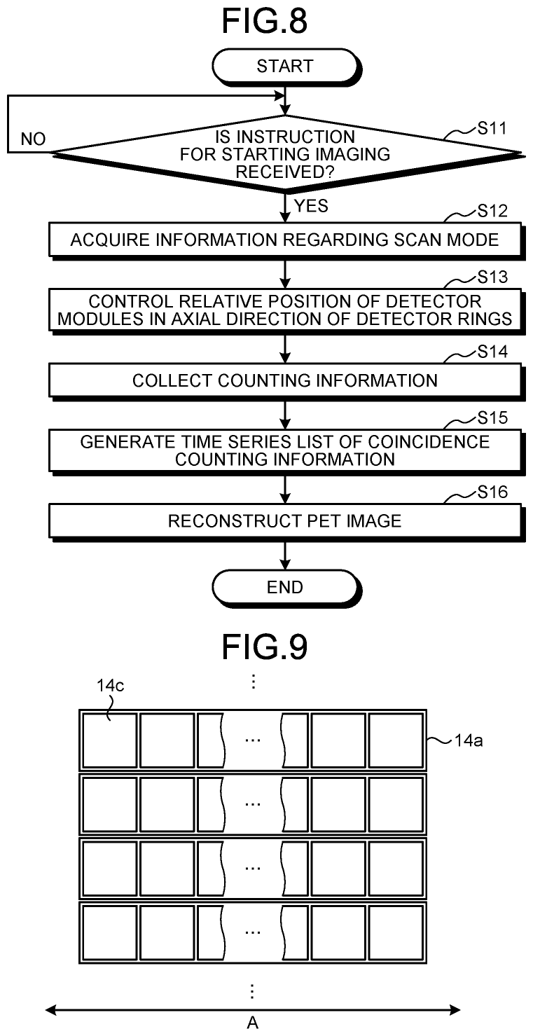

FIG. 8 is a flowchart illustrating an order of the processing executed by the processing circuitry 24 of the PET apparatus 100 according to the first embodiment.

For example, as illustrated in FIG. 5, the processing circuitry 24 executes the following processing when the subject P is placed in the opening part of the gantry 10 and an instruction for starting imaging is received from the operator (Yes at step S11).

First, the processing circuitry 24 acquires the information regarding the scan mode of the PET scan for the subject P (step S12). This step is a step corresponding to the acquisition function 24d. For example, the processing circuitry 24 executes the step by reading out and executing the computer program corresponding to the acquisition function 24d from the memory 23.

Subsequently, the processing circuitry 24 controls the relative position of the detector modules 14c in the axial direction of the detector rings 14b based on the acquired information regarding the scan mode (step S13). This step is a step corresponding to the control function 24c. For example, the processing circuitry 24 executes the step by reading out and executing the computer program corresponding to the control function 24c from the memory 23.

Subsequently, the processing circuitry 24 controls the counting information collection circuitry 15 to collect the counting information on the pair annihilation gamma rays emitted from the subject P (step S14). This step is a step corresponding to the control function 24c. For example, the processing circuitry 24 executes the step by reading out and executing the computer program corresponding to the control function 24c from the memory 23.

Subsequently, the processing circuitry 24 generates a time series list of coincidence counting information by using the counting information collected by the counting information collection circuitry 15 (step S15). This step is a step corresponding to the coincidence counting information generation function 24a. For example, the processing circuitry 24 executes the step by reading out and executing the computer program corresponding to the coincidence counting information generation function 24a from the memory 23.

Subsequently, the processing circuitry 24 reconstructs a PET image of the subject P based on the generated time series list of the coincidence counting information (step S16). This step is a step corresponding to the image reconstruction function 24b. For example, the processing circuitry 24 executes the step by reading out and executing the computer program corresponding to the image reconstruction function 24b from the memory 23.

As described above, in the first embodiment, the PET detector 14 includes the detector rings configured with the detector modules arranged in an annular shape. Furthermore, the acquisition function 24d of the processing circuitry 24 acquires the information regarding the scan mode of the PET scan for the subject P. Moreover, the control function 24c of the processing circuitry 24 controls the relative position of the detector modules in the axial direction of the detector rings based on the acquired information regarding the scan mode acquired by the acquisition function 24d.

With such a configuration, it is possible to perform imaging with the AFOV adjusted to an appropriate size in accordance with the scan mode of the PET scan. Thus, in a case where imaging is performed for a plurality of times by moving the couchtop 11 on which the subject P is placed as in a case of whole-body imaging, for example, it is possible to reduce the number of times of imaging, thereby reducing the entire imaging time. Therefore, according to the first embodiment, the imaging throughput can be improved.

Note that it is also considered to use a PET apparatus that is lengthy in the axial direction as a configuration for improving the imaging throughput, for example. In that case, however, still more detection elements are required, so that the cost for the PET apparatus is greatly increased. On the contrary, it is possible with the first embodiment to improve the imaging throughput at a lower cost compared to the case of using the PET apparatus that is lengthy in the axial direction.

While the first embodiment has been described heretofore, a part of the configuration of the above-described PET apparatus 100 may be modified as appropriate. Thus, a modification example regarding the first embodiment will be described hereinafter as another embodiment. Note that the embodiment will be described hereinafter concentrating mainly on different points with respect to the first embodiment, and the points duplicated with the already-described content will not be described in detail.

### Second Embodiment

Furthermore, for example, the PET apparatus 100 described above may be further configured to control the relative position in the axial direction of the detector modules 14c included in each of the detector units 14a. Hereinafter, such an example will be described as the second embodiment.

In this embodiment, the detector drive mechanism 16 individually moves not only the detector units 14a included in the PET detector 14 but also the detector modules 14c included in each of the detector units 14a.

Furthermore, in this embodiment, the control function 24c controls the relative position of the detector units 14a in the axial direction of the detector rings 14b as described in the embodiments above and controls the relative position in the axial direction of the detector modules 14c included in each of the detector units 14a.

For example, as in the first embodiment, the control function 24c controls the shift amount of the detector units 14a included in the first group and the detector units 14a included in the second group with the three modes that are the normal mode, the wide mode, and the intermediate mode.

FIG. 9 to FIG. 11 are diagrams illustrating examples of control of the relative position of the detector modules 14c performed by the control function 24c according to the second embodiment.

For example, as illustrated in FIG. 9, the control function 24c in the normal mode controls the detector drive mechanism 16 so that the positions of the detector units 14a included in the first group coincide with the positions of the detector units 14a included in the second group in the axial direction of the PET detector 14 as in the first embodiment.

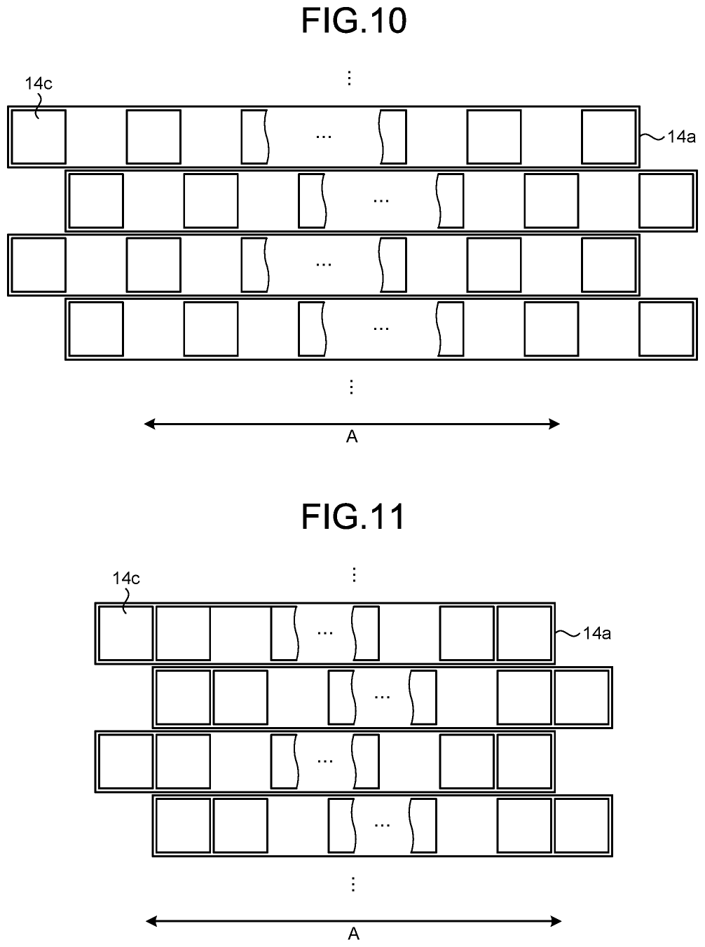

In the meantime, as illustrated in FIG. 10, for example, the control function 24c in the wide mode controls the detector drive mechanism 16 such that the detector modules 14c are disposed with a space of the size of a single detector module 14c being provided for every detector module 14c within each of the detector units 14a. Furthermore, the control function 24c controls the detector drive mechanism 16 so that the detector units 14a included in the first group and the detector units 14a included in the second group are in a positional relationship being shifted from each other by the size of a single detector module 14c. Thus, in the PET detector 14, the detector modules 14c are disposed alternately along the axial direction and the circumferential direction, respectively.

Furthermore, as illustrated in FIG. 11, for example, the control function 24c in the intermediate mode controls the detector drive mechanism 16 such that the detector modules 14c are disposed with a space of the size of a single detector module 14c being provided for every two detector modules 14c within each of the detector units 14a. Furthermore, the control function 24c controls the detector drive mechanism 16 such that the detector units 14a included in the first group and the detector units 14a included in the second group are in a positional relationship being shifted from each other by the size of a single detector module 14c. Note that the shift amount of the detector units 14a and the layout of the detector modules 14c within the detector units 14a in the intermediate mode may be changed arbitrarily in accordance with the operation of the operator, for example.

As described above, in the second embodiment, the control function 24c controls not only the relative position of the detector units 14a in the axial direction of the detector rings 14b but also the relative position in the axial direction of the detector modules 14c included in each of the detector units 14a.

With such a configuration, it is possible to dispose the detector modules 14c in a dispersed manner in the axial direction in the PET detector 14, so that the spatial resolution within the AFOV can be uniform.

### Third Embodiment

Furthermore, for example, the PET apparatus 100 described above may be further configured to rotate the detector rings 14b about the center axis or to move the detector rings 14b in the axial direction so that the data in the gap part caused by the shift of the detector modules 14c is compensated. Hereinafter, such an example will be described as the third embodiment.

In the embodiment, the detector drive mechanism 16 not only moves the detector units 14a included in the PET detector 14 individually but also rotates the detector rings 14b about the center axis or to move the detector rings 14b in the axial direction.

Furthermore, in this embodiment, the control function 24c controls the relative position of the detector modules 14c in the axial direction of the detector rings 14b as described in the embodiments above and rotates the detector rings 14b about the center axis or moves the detector rings 14b in the axial direction so that the data in the gap part caused by the shift of the detector modules 14c is compensated.

For example, when imaging is performed with the wide mode or the intermediate mode, the control function 24c rotates the detector rings 14b about the center axis after collecting the coincidence counting information once so as to move the detector units 14a included in the PET detector 14 for the size of a single detector module 14c in the circumferential direction. Then, the control function 24c collects the coincidence counting information again after completing the move of the detector units 14a. Thus, the data in the gap part caused by the shift of the detector modules 14c is compensated.

Alternatively, for example, when imaging is performed with the wide mode or the intermediate mode, the control function 24c moves the detector rings 14b in the axial direction after collecting the coincidence counting information once so as to move the detector units 14a included in the PET detector 14 in the axial direction for the size of a single detector unit 14a. Then, the control function 24c collects the coincidence counting information again after completing the move of the detector units 14a. Thus, the data in the gap part caused by the shift of the detector modules 14c is compensated.

With such a configuration, it is possible to improve the image quality of the PET images by compensating the data in the gap part caused by the shift of the detector modules 14c.

### Fourth Embodiment

Furthermore, for example, the PET apparatus 100 described above may be further configured to continuously move the couchtop 11 on the inner side of the detector rings 14b to perform imaging while rotating or moving the detector rings 14b. Hereinafter, such an example will be described as the fourth embodiment.

In this embodiment, the control function 24c controls the relative position of the detector modules 14c in the axial direction of the detector rings 14b as described in the embodiment above, and furthermore the control function 24c continuously moves the couchtop 11 on which the subject P is placed in the axial direction on the inner side of the detector rings 14b to perform imaging while rotating the detector rings 14b about the center axis.

For example, when imaging of a whole body of the subject P is performed, the control function 24c controls the detector drive mechanism 16 and the couch drive mechanism 13 while controlling the relative position of the detector modules 14c at the respective positions in the axial direction as described in the embodiment above so as to continuously move the couchtop 11 on which the subject P is placed in the axial direction while continuously rotating the detector rings 14b about the center axis. At this time, furthermore, the control function 24c may continuously move the detector rings 14b in a direction opposite from the direction to which the couchtop 11 is moved in the axial direction of the detector rings 14b.

With such a configuration, imaging of the whole body of the subject P can be performed efficiently, so that the imaging throughput can be further improved.

### Fifth Embodiment

Furthermore, for example, the PET apparatus 100 described above may be further configured to correct the collected data so that deterioration of the spatial resolution caused in the peripheral edges and the like of the AFOV by the shift of the detector modules 14c is compensated. Hereinafter, such an example will be described as the fifth embodiment.

In this embodiment, the image reconstruction function 24b corrects the collected data so that deterioration of the spatial resolution caused by the shift of the detector modules 14c in the axial direction of the detector ring 14b is compensated. Note that the image reconstruction function 24b is an example of the correction unit.

For example, the image reconstruction function 24b corrects the data by using a trained model to which input of the data collected while the detector units 14a are being shifted is input and which outputs corrected data. In this case, for example, the trained model is built by machine learning having the data that is collected while the detector units 14a are being shifted (the wide mode, the intermediate mode) and the data collected by moving the couchtop with the normal mode as learning data. Note that the data as the target of correction may be the coincidence counting information or may be a PET image reconstructed from the coincidence counting information.

With such a configuration, it is possible to improve the image quality of the PET image by correcting the data so that deterioration of the spatial resolution caused by the shift of the detector modules 14c is compensated.

### Other Embodiment

While the control function 24c in the embodiments above is described to move the detector units 14a included in the PET detector 14 in a unit of two groups to which the detector units 14a are distributed alternately along the circumferential direction, the embodiments are not limited thereto. For example, the control function 24c may move the detector units 14a in a unit of three or more groups to which every two pieces or more of the detector units 14a are distributed along the circumferential direction. In this case, the range capable of disposing the detector modules 14c can be further expanded, so that the maximum AFOV can be further increased compared to the case with the two groups.

Furthermore, the configuration of the PET apparatus 100 described in the embodiments above is not limited to be applied to a single PET apparatus. For example, the configuration of the PET apparatus 100 described in the embodiments above can be applied to a PET-CT apparatus in which a PET apparatus and an X-ray CT apparatus are combined, and a PET-Magnetic Resonance Imaging (MRI) apparatus in which a PET apparatus and an MRI apparatus are combined.

Furthermore, the processing circuitry in the embodiments above is not limited to a type implemented by a single processor but may also be processing circuitry configured by combining a plurality of independent processors and each of the processors may implement each of the processing functions by executing a computer program. Furthermore, each of the processing functions of the processing circuitry may be implemented by being distributed or integrated to a single or a plurality of processing circuitries as appropriate. Furthermore, each of the processing functions of the processing circuitry may be implemented by a mixture of hardware such as circuitry and software. While an example of the case where the computer programs corresponding to each of the processing functions are stored in a single memory has been described herein, the embodiments are not limited thereto. For example, the computer programs corresponding to each of the processing functions may be distributed and stored in a plurality of memories, and each of the computer programs may be read out from each of the memories to be executed.

Furthermore, while the embodiments above are described by referring to the examples of the case where the control unit and the correction unit of the current Description are implemented by the control function 24c and the image reconstruction function 24b of the processing circuitry 24, the embodiments are not limited thereto. For example, in addition to the case implementing the control unit and the correction unit of the current Description by the control function 24c and the image reconstruction function 24b described in the embodiments, the same functions may be implemented by hardware alone, software alone, or a mixture of hardware and software.

Furthermore, the term “processor” used in the explanation of the embodiments above means a Central Processing Unit (CPU), a Graphics Processing Unit (GPU), or a circuit such as an Application Specific Integrated Circuit (ASIC), a programmable logic device (for example, a Simple Programmable Logic Device: SPLD), a Complex Programmable Logic Device (CPLD), a Field Programmable Gate Array (FPGA), and the like. Note that the computer programs may directly be embedded in the circuit of the processor instead of saving the computer programs in the memory. In this case, the processor implements the functions by reading out and executing the computer programs embedded in the circuit. Furthermore, each of the processors of this embodiment is not limited to be configured as a single circuit for each processor but may be configured as a single processor by combining a plurality of independent circuits to implement the functions thereof.

Note that the computer programs to be executed by the processor are embedded in a Read Only Memory (ROM) or the like in advance to be provided. The computer programs may be provided as electronic files in a format installable to or executable by those devices that is recorded in non-transitory computer readable storage media such as a Compact Disc (CD)-ROM, a Flexible Disk (FD), a CD-Recordable (CD-R), and a Digital Versatile Disc (DVD) in a file of format installable to or executable by those devices. Furthermore, the computer programs may be stored on a computer connected to a network such as the Internet, and may be provided or distributed by being downloaded via the Internet. For example, such a computer program is configured by modules including each of the above-described processing functions. As the actual hardware, the CPU reads out and executes the computer program from the storage medium such as the ROM, so that each of the modules is loaded and generated on a main memory.

Furthermore, each of the apparatuses and structural elements illustrated in the embodiments is the functional concept thereof, and not necessarily needs to be physically configured as illustrated. That is, specific forms of distribution or integration of each of the apparatuses are not limited to those as illustrated, and a whole part or a part thereof can be functionally or physically distributed or integrated in any desired unit in accordance with various kinds of load, use conditions, and the like. Furthermore, a whole part or a part of each of the functions executed by each of the apparatuses may be implemented by a CPU or a computer program analyzed and executed by the CPU or may be implemented as hardware with wired logic.

Furthermore, as for each processing described in the embodiments above, a whole part or a part of the processing described to be executed automatically may be executed manually or a whole part or a part of the processing described to be executed manually may be executed automatically by a known method. In addition, the processing order, the control order, the specific names, and the information including various kinds of data and parameters presented in the explanation above and the drawings can be changed arbitrarily unless otherwise noted.

According to at least one of the embodiments described above, the imaging throughput can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A positron emission tomography (PET) apparatus comprising:
a PET detector including a detector ring configured with a plurality of detector modules arranged in an annular shape; and
processing circuitry configured to
acquire information regarding a scan mode of a PET scan for a subject, and
control a relative position of the detector modules in an axial direction of the detector ring based on the information.

2. The positron emission tomography apparatus according to claim 1, wherein the processing circuitry is further configured to
acquire information regarding a body site of the subject as the information regarding the scan mode, and
control the relative position of the detector modules so that a shift amount of the detector modules in the axial direction changes in accordance with the body site.

3. The positron emission tomography apparatus according to claim 1, wherein the processing circuitry is further configured to
acquire attenuation data indicating a distribution of attenuation coefficients in a body of the subject as the information regarding the scan mode, and
control the relative position of the detector modules so that counted number of pair annihilation gamma rays detected by the PET detector at each position along the axial direction becomes uniform.

4. The positron emission tomography apparatus according to claim 1, wherein
the PET detector includes a plurality of the detector rings arranged in the axial direction with respective center axes being aligned with each other,
the detector rings are configured with a plurality of detector units arranged in an annular shape, the detector units each being configured with the detector modules as many as the detector rings and arranged in the axial direction, and
the processing circuitry is further configured to control a relative position of the detector units in the axial direction to control the relative position of the detector modules.

5. The positron emission tomography apparatus according to claim 4, wherein the processing circuitry is further configured to control the relative position in the axial direction of the detector modules included in each of the detector units.

6. The positron emission tomography apparatus according to claim 1, wherein the processing circuitry is further configured to rotate the detector ring about a center axis or move the detector ring in the axial direction so that data in a gap part caused by shift of the detector modules in the axial direction is compensated.

7. The positron emission tomography apparatus according to claim 1, wherein the processing circuitry is further configured to continuously move a couchtop on which the subject is placed in the axial direction on an inner side of the detector ring to perform imaging while rotating the detector ring about a center axis.

8. The positron emission tomography apparatus according to claim 1, wherein the processing circuitry is further configured to correct collected data so that deterioration of spatial resolution caused by shift of the detector modules in the axial direction is compensated.
